(19) United States
(12) Patent Application Publication
(10) Pub. No.: US 2009/0192511 A1
(43) Pub. Date: Jul. 30, 2009

(54) Surgical instruments and instrument handle having support brace

(76) Inventor: Mark E. Haffenreffer, Dedham, MA (US)

Correspondence Address: MUIRHEAD AND SATURNELLI, LLC, 200 FRIBERG PARKWAY, SUITE 1001, WESTBOROUGH, MA, 01581, US

(21) Appl. No.: 12/011,593

(22) Filed: Jan. 28, 2008

(51) Int. Cl. A61B 17/56

(52) U.S. Cl. 606/53

(57) ABSTRACT

Orthopedic procedures, such as total hip replacement and/or other surgical procedures, involve the retraction and distraction of bone and tissue in order to reduce prosthesis components, such as a femoral head prosthesis, into place during surgery and to perform other surgical techniques, such as reaming of the acetabulum. An instrument handle that allows for enhanced control of surgical instruments and other tools that involve the application of directional force includes a grip, an extension arm and a support brace that is configured to contact a user's forearm during a surgical procedure. A multiple-tool receptor may also be included that allows for the removable and interchangeable use of tools in connection with the instrument handle. The system described herein allows for deep distraction without angulation using larger muscles or even one's body.

### TECHNICAL FIELD

This application relates to the field of instruments and handles therefor, and more particularly to the field of surgical instruments.

### BACKGROUND OF THE INVENTION

Orthopedic procedures, such as total hip replacement and/or other surgical procedures, involve the retraction and distraction of bone and tissue in order to reduce prosthesis components, such as a femoral head prosthesis, into place during surgery and to perform other surgical techniques, such as reaming of the acetabulum, which is the socket into which a femur head is disposed. Retraction and distraction techniques involve the application of force to bone and/or tissue to exposure surgical areas and may require a sizable amount of force for an extended period of time. The use of femoral head prostheses of larger sizes to reduce the changes of dislocation further increases the amount of force required since greater distraction of components is required.

Known retractor instruments used in orthopedic procedures often require the use of hand and wrist muscles to control angulation and rotation to reduce components into place during the surgery and thereby lessens the amount of power a doctor may comfortably be able to employ for retraction and/or distraction procedures. In particular, the required control of angulation and rotation of the orthopedic instruments increases the difficulty of reducing components into place and increases the chances that the components will engage before the reduction and possibly become scratched. Scratching causes wear which may ultimately lead to loose components and necessitate a future remedial operation.

U.S. Pat. No. 5,379,758 to Snyder entitled “Hand Held Surgical Retractor”, which is incorporated herein by reference, discloses a surgical retractor having a blade disposed on one end of a handle and a brace plate attached to an opposite free end that fits against the user's forearm to stabilize the retractor. A hand grip is attached to the handle between the blade and brace plate. A fulcrum is created at the attachment between the hand grip and the handle. It should be noted that as a result of the fulcrum attachment, the grip is movable with respect to the handle and the blade of the retractor, the grip and the brace plate are all serially configured in a line approximately parallel to the user's forearm, which, in some cases, may not allow for the exertion of sufficient directional force, and the control thereof, that may be necessary for some surgical procedures.

Accordingly, it would be desirable to provide a system that allows for enhanced control of surgical instruments and other tools that involve the application of directional force.

### SUMMARY OF THE INVENTION

According to the system described herein, an instrument handle includes a grip, an extension arm coupled to a first end of the grip, and a support brace coupled to the extension arm. A multiple-tool receptor is coupled to a second end of the grip, wherein the multiple-tool receptor includes an interface for removably attaching a tool. The interface may be coupled to the second end of the grip at a position opposite from the first end of the grip coupled to the extension arm. The interface may have a tool-receiving axis that is substantially perpendicular to the extension arm. The support brace may have a U shape with at least one arm being adjustable in length. The extension arm may be adjustable in length. The support brace may be movable on the extension arm. The multiple-tool receptor may include at least one of: a bushing; a quick connect mechanism; a shank; and a chuck. A swivel mechanism may be coupled to the grip, the swivel mechanism including a opening for coupling to a body linkage device. The grip may be angled with respect to the extension arm.

According further to the system described herein, a surgical instrument an instrument handle and a tool coupled to the instrument handle. The instrument handle includes a grip, an extension arm coupled to a first end the grip, and a support brace coupled to the extension arm, wherein the tool is coupled to a second end of the grip at a position opposite from the first end of the grip coupled to the extension arm. A multiple-tool receptor may be coupled to the second end of the grip, wherein the multiple-tool receptor includes an interface for removably attaching the tool. The tool may include a femoral distractor hook, a femoral grasp and/or a retractor. The instrument handle may further include a swivel mechanism coupled to the grip, the swivel mechanism including a opening for coupling to a body linkage device. The interface may have a tool-receiving axis that is substantially perpendicular to the extension arm. At least one of the extension arm and the support brace may be adjustable in length or position.

According further to the system described herein, a method for performing a hip replacement procedure includes positioning a user's forearm in an instrument handle of a surgical instrument, the instrument handle including a grip, an extension arm a support brace that contacts the forearm. A first tool of the surgical instrument extends from the grip in a direction substantially perpendicular to the extension arm. Force is exerted on the surgical instrument in a direction substantially parallel to the extension arm, wherein the support brace braces against the forearm as a result of the force exerted. The first tool may be removed from the grip of the instrument handle and a second tool inserted into the grip of the surgical handle. Bracing of the support brace against the forearm may minimize angulation and rotation of the surgical instrument.

The system described herein allows for deep distraction without angulation using larger muscles or even one's body.

### BRIEF DESCRIPTION OF THE DRAWINGS

Embodiments of the system are described with reference to the several figures of the drawings, in which:

FIG. 1A is a schematic illustration of an instrument handle with a grip, an extension arm, a support brace and a multi-tool receptor fitting according to an embodiment of the system described herein.

FIG. 1B is a schematic illustration of the instrument handle of FIG. 1A as grasped by a user and positioned on the user's forearm according to an embodiment of the system described herein.

FIGS. 2A and 2B are schematic illustrations of an instrument handle having an angled grip according to various embodiments of the system described herein.

FIG. 3 is a schematic illustration showing a pistol-type grip of an instrument handle according to an embodiment of the system described herein.

FIG. 4 is a schematic illustration showing an instrument handle having adjustable components according to an embodiment of the system described herein.

FIGS. 5A and 5B are schematic illustrations of hook femoral distractor/elevation instrument each including an instrument handle with multi-tool receptor and a femoral distractor hook component removably fitted therein according to various embodiments of the system described herein.

FIGS. 6A and 6B are schematic illustrations of hook femoral distractor instruments each including an instrument handle and a femoral distractor hook component permanently attached thereto according to various embodiments of the system described herein.

FIGS. 7A, 7B, 7C, 8A and 8B illustrate other example retractor tools having cylindrical and non-cylindrical tool shank shapes that may be used in connection with the system described herein.

FIGS. 9A and 9B are schematic illustrations of a femoral grasp tool that may be used with an instrument handle in accordance with the system described herein.

FIG. 10 is a schematic illustration of a swivel body mechanism for use with an instrument handle in accordance with the system described herein.

FIG. 11 is schematic illustration of a body linkage device coupled to a swivel body mechanism for use with an instrument handle in accordance with the system described herein.

### DETAILED DESCRIPTION OF VARIOUS EMBODIMENTS

Referring now to the figures of the drawings, the figures comprise a part of this specification and illustrate exemplary embodiments of the described system. It is to be understood that in some instances various aspects of the system may be shown schematically or may be exaggerated or altered to facilitate an understanding of the system.

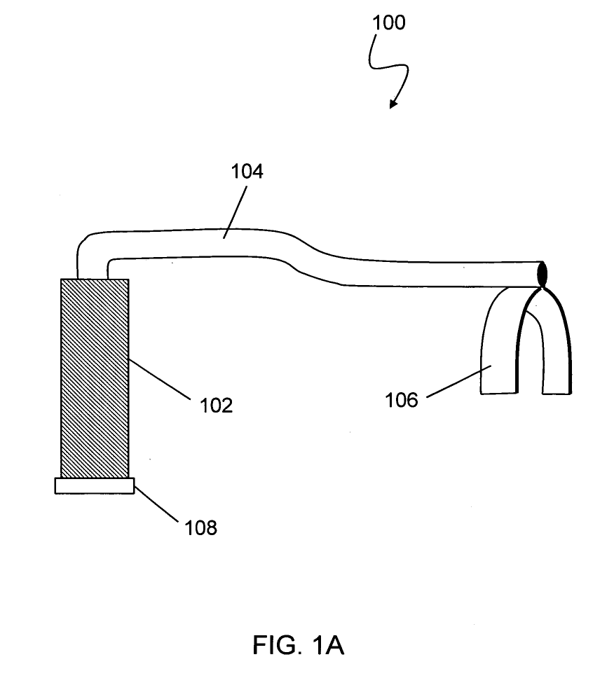

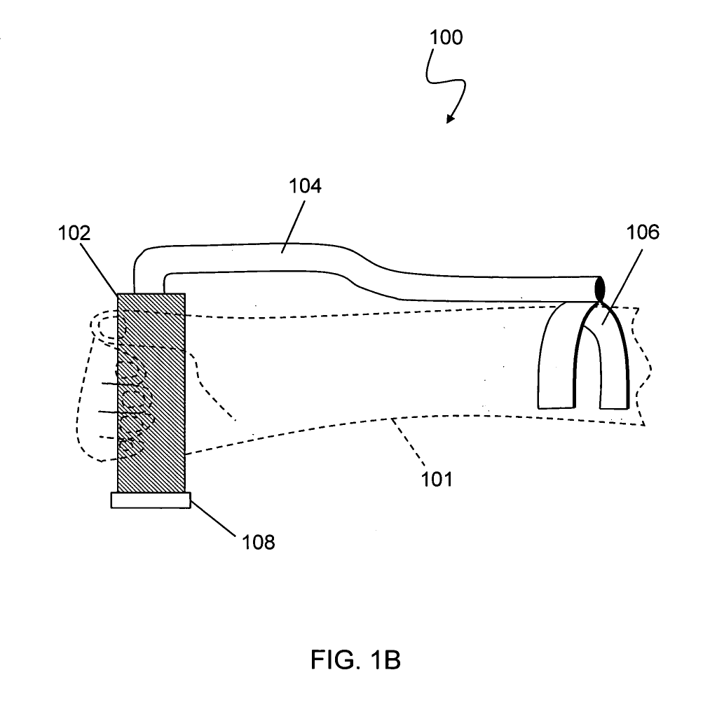

FIG. 1A is a schematic illustration of an instrument handle 100 according to an embodiment of the system described herein. The handle 100 includes a grip 102, an extension arm 104 attached to the grip 102 and a support brace 106 attached at the end of the extension arm 104. The end of the grip 102 may include a multiple-tool receptor 108, such as a bushing and/or other universal-type joint interface, that allows multiple tool components to be removably fitted therein. For example, the multiple-tool receptor 108 may include: a quick-connect mechanism in which a sleeve is pulled up to release the end and drops down to lock a tool into place; a shank assembly, such as a Trinkle shank; and/or a chuck assembly, such as a Jacob's chuck. In an embodiment, a hook femoral distractor tool may be fitted into the multiple-tool receptor 108, among other components, as further described elsewhere herein. In an embodiment, the grip 102 and receptor 108 are disposed to allow a surgical tool component to extend substantially perpendicular to the extension arm and/or a user's forearm.

The support brace 106 attached at the end of the extension arm 104 may be positioned contact the forearm of a user holding the grip 102 of the handle 100. In this way, the handle 100 may control rotation and angulation of a surgical tool, such as a femoral distractor. The support brace 106 of the handle 100 allows a doctor to use his or her tricep muscles, rather than only forearm, wrist and finger muscles, when exerting a directional force, such as that required for distraction of components in a total hip replacement procedure to reduce components of a prosthesis in to place during surgery. This allows the doctor to exert more force in a controllably way thereby facilitating the reducing of the prosthesis components into place and making it easier to prevent scratching of the components. The distraction of components and material, such as tissue, rather than angulation thereof, allows for improved exposure of the site of interest.

FIG. 1B a schematic illustration of the instrument handle 100 of FIG. 1A as positioned on the user's forearm 101 according to an embodiment of the system described herein. As illustrated, the support brace 106 that is disposed at the end of the extension arm 104 may be designed to contact the user's forearm 101 when the user grasps the grip 102 to provide the enhanced force and control as discussed herein. In various embodiments, the handle 100 may be constructed of metal, such as surgical grade stainless steel and/or other suitable material that is strong enough to allow the user to exert sufficient force to perform surgical procedures, such as femoral distraction or retraction procedures. Flexible straps may also be used in connection with the grip 106 and/or with the support brace 102. Further, the support brace 102 may have a semi-circular cuff shape, a circular shape into which a user's arm is inserted and/or other shape that allows the handle to be braced against a user's arm.

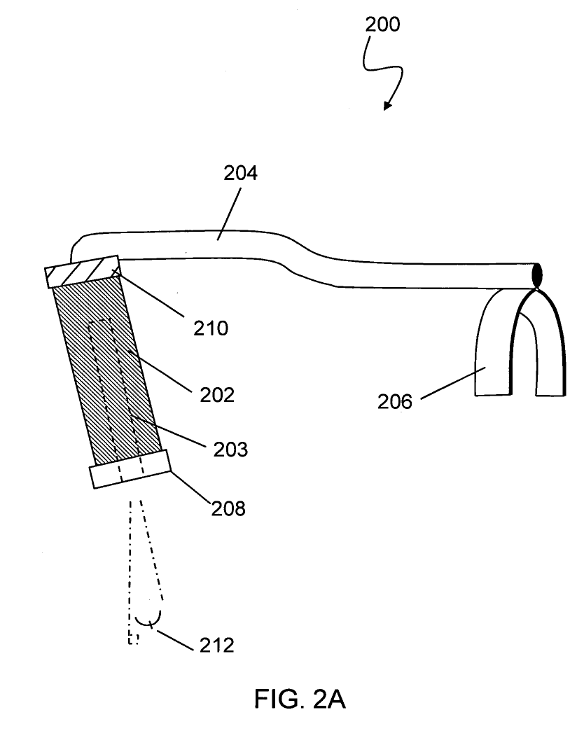

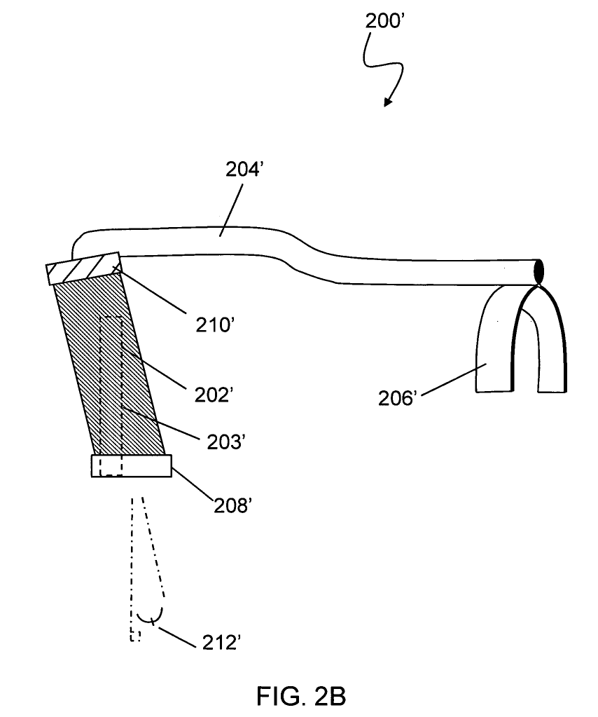

FIGS. 2A and 2B are schematic illustrations of an instrument handle 200, 200′ having an angled grip 202, 202′ according to various embodiments of the system described herein. As shown in FIG. 2A, the handle 200 may include an extension arm 204, support brace 206 and multiple-tool receptor 208 that are like similar components discussed herein, such as the components 104, 106, 108. The angled grip 202 may be angled in an ergonomic way to enhance the comfort and functionality of a user grasping the grip 202. For example, an angle 212 of the angled grip 202 may be in a range from zero to thirty degrees and/or specifically angled at about fifteen degrees which may be the natural incline from the little finger to the index finger, upward, for the wrist and forearm. The angle 212 may be customized for a user and/or may be adjustable with an adjustable interface 210 that may allow manipulation of the angle 212 and the locking of the angled grip 202 at the desired position. As illustrated, the angled grip 202 is shown containing a cavity 203 into which a portion of a tool may be inserted and locked into place with the multiple-tool receptor 208. A tool-receiving axis of the multiple-tool receptor 208 and cavity 203 may be parallel to the angle 212 of the angled grip. In this case, in order to maintain a tool axis that is substantially parallel to the extension arm 204, if desired, different tool shank shapes may be used, such as tool shanks a bend, as further discussed elsewhere herein. Alternatively, other grip and tool receptor designs may be used, as further discussed, for example, in connection with FIG. 2B.

FIG. 2B is a schematic illustration of another embodiment of an instrument handle 200′ having an angled grip 202′. The handle 200′ may include similar components as discussed in connection with the handle 200, including an extension arm 204′, support brace 206′ and multiple-tool receptor 208′. However, as shown in the figure, in the illustrated embodiment, the angled grip 202′ may include a cavity 203′ that is oriented substantially perpendicular to the extension arm 204′ despite the angle 212′ of the angled grip 202′. Similarly, the multiple-tool receptor 208′ may have a tool-receiving axis that is substantially perpendicular to the extension arm 204′. In this way, no bends in the insertion tool shanks may be required allowing the same tool shank designs to be used for angled or unangled grips. Other angles of the tool-receiving axis may be used in other circumstances, for example, depending on the type of tool to be inserted into the handle and the requirements of the instrument for a surgical procedure.

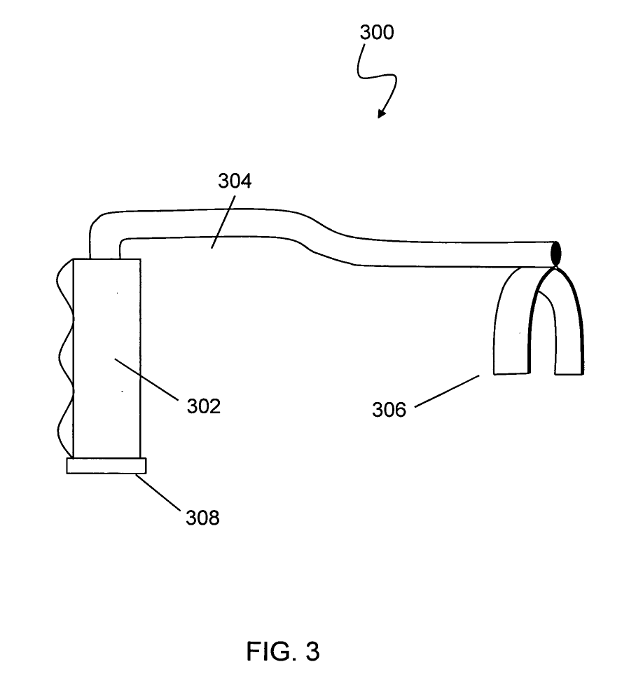

FIG. 3 is a schematic illustration showing a shaped grip 302 of an instrument handle 300 according to an embodiment of the system described herein. The shaped grip 302 may be a pistol-type grip and/or other ergonomically-designed grip that facilitates the holding of the grip. The shaped grip 302 may also be angled and include a textured surface, as further discussed elsewhere herein in connection with other embodiments. The handle 300 may include an extension arm 304, support brace 306 and multiple-tool receptor 308 that are like similar components discussed herein, such as the components 104, 106, 108.

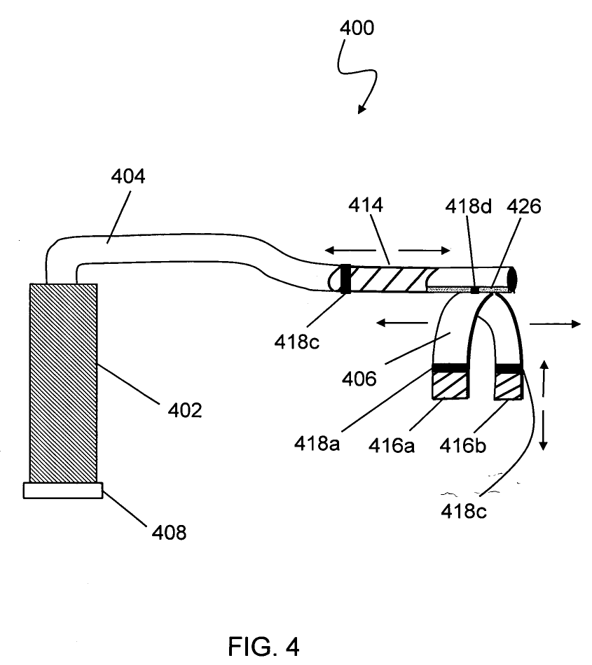

FIG. 4 is a schematic illustration showing an instrument handle 400 having adjustable components according to an embodiment of the system described herein. The handle 400 may include a grip 402, an extension arm 404, a support brace 406 and a multiple-tool receptor 408. As illustrated, the support brace 406, functioning like the support brace 106 discussed elsewhere herein, may include adjustable arms 416a, 416b that may be adjusted to increase and/or decrease the arm lengths of the support brace 106, including extension in a curved fashion in which the arm 416a is joined with the arm 416b to form a completely enclosed support brace. The adjustable arms 416a, 416b may be locked into place using one or more locking mechanisms 418a, 418b included therewith, such as a locking nut, locking screw and/or other locking device. An extension arm 404, like the extension arm 104 discussed elsewhere herein, may include an adjustment mechanism 414 that allows the extension arm 404 to be lengthened and/or shortened, and then locked into position using a locking mechanism 418c, such as a locking nut, locking screw and/or other locking device, according to the requirements of a user. Further, the support brace 406 may be coupled to the extension arm 406 by a sliding mechanism 426 that allows the support brace 406 to be slidably positioned along the extension arm 404 and then locked into place using a locking mechanism 418d, such as a locking nut, locking screw and/or other locking device, at a desired position. The various adjustment mechanisms described in connection with the handle 400 may be included individually or in any combination.

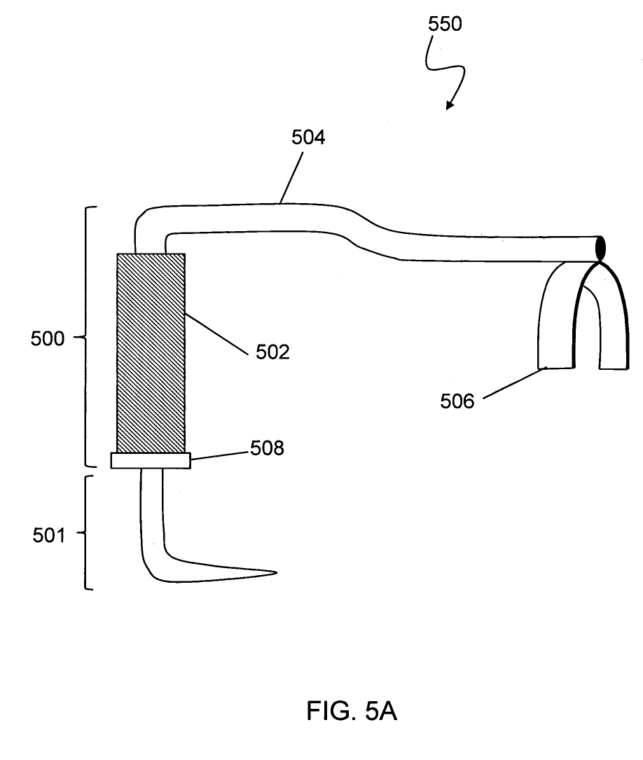

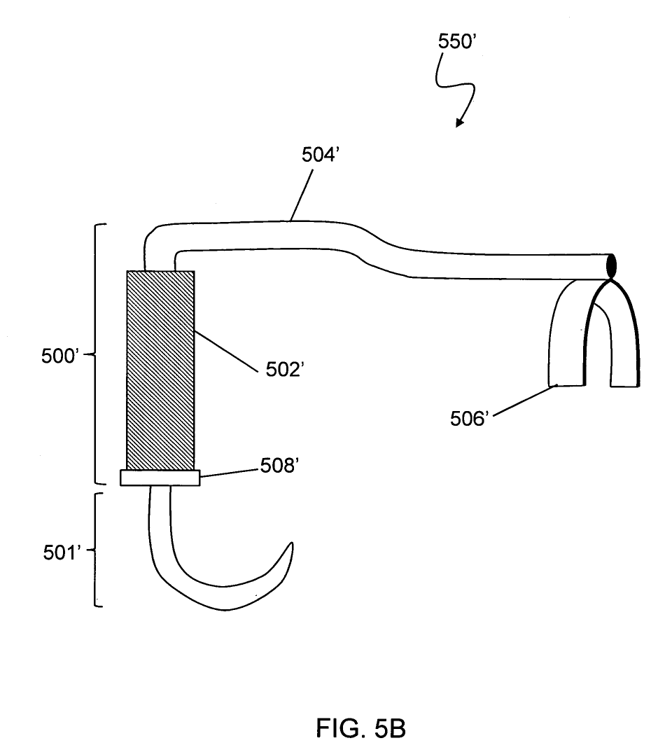

FIGS. 5A and 5B are schematic illustrations hook femoral distractor/elevation instruments 550, 550′ including instrument handles 500, 500′ and femoral distractor/elevation hook tools 501, 501′ removably fitted therein according to an embodiment of the system described herein. The instrument 550 may be used for distraction while the instrument 550′ may be used for elevation. As shown in FIG. 5A, the instrument 550 may include an instrument handle 500 with a grip 502, an extension arm 504, a support brace 506 and a multiple-tool receptor 508 that function like the similar components 102, 104, 106, 108 discussed elsewhere herein. The multiple-tool receptor 508 receives an end of a femoral distractor/elevation hook tool 501 which is locked into place. In other embodiments the other tools may be used in connection with the instrument handle 500, as discussed elsewhere herein. As further discussed elsewhere herein, various known mechanisms for the multiple-tool receptor 508 may be used including quick-connect mechanisms, shank assemblies and/or chuck assemblies, among others. Note that, generally, the length of the shank of the tool 501 of FIG. 5A, or the length of the shank of any other tool, may be adjusted to a length that is appropriate for the type of tool, its use, and/or possibly the expected user of the tool.

FIG. 5B illustrates another embodiment of a femoral distractor/elevation hook tool 501′ having a more pronounced hook at the end thereof, such as a bone hook, than is shown in connection with femoral distractor/elevation hook 501 discussed elsewhere herein. The instrument 550′ may include an instrument handle 500′ with a grip 502′, an extension arm 504′, a support brace 506′ and a multiple-tool receptor 508′ that function like the similar components discussed in connection with the instrument 550.

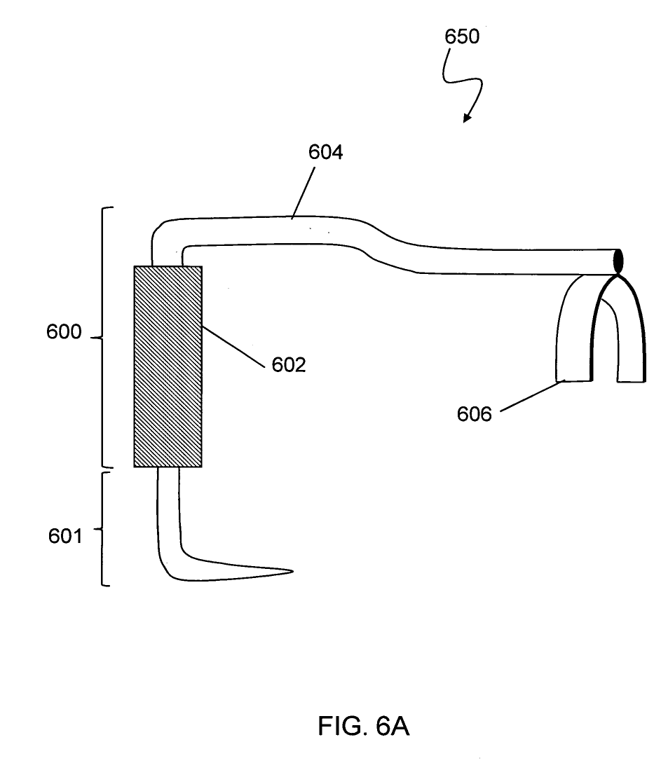

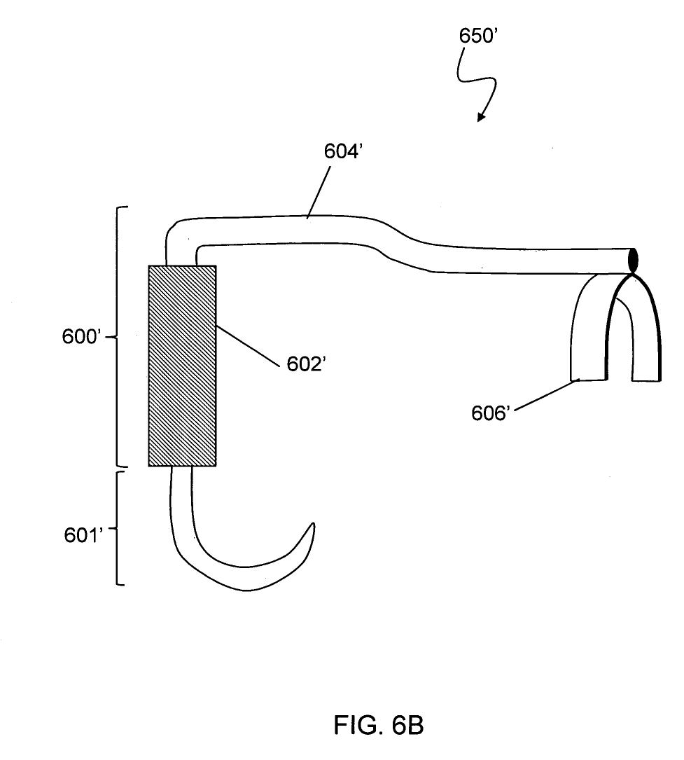

FIGS. 6A and 6B are schematic illustration of hook femoral distractor instruments 650, 650′ including instrument handles 600, 600′ and femoral distractor hook tools 601, 601′ permanently attached thereto according to an embodiment of the system described herein. Permanent attachment may include welding and/or other joining process in which the tool head is not easily removable from the handle or interchangeable with other tools. As shown in FIG. 6A, the instrument 650 may include the instrument handle 600 with a grip 602, an extension arm 604, and a support brace 606 that function like the similar components 602, 604, 606, discussed elsewhere herein. The permanent joint between the femoral distractor hook tool 601 and the instrument handle 600 may allow for an instrument having enhanced strength that is especially suitable for force-intensive surgical procedures and including components for enhanced control in accordance with the features and functions of the embodiments of the system discussed herein. Other instruments may be manufactured using other types of tools permanent attached to the handle 600. Any of the other tools/instruments described attachments herein may also be permanently attached.

FIG. 6B illustrates another embodiment of a femoral distractor hook tool 601′ having a more pronounced hook at the end thereof, such as a bone hook, than is shown in connection with femoral distractor hook 601 discussed elsewhere herein. The instrument 650′ may include an instrument handle 600′ with a grip 602′, an extension arm 604′ and a support brace 606′ that function like the similar components discussed in connection with the instrument 650.

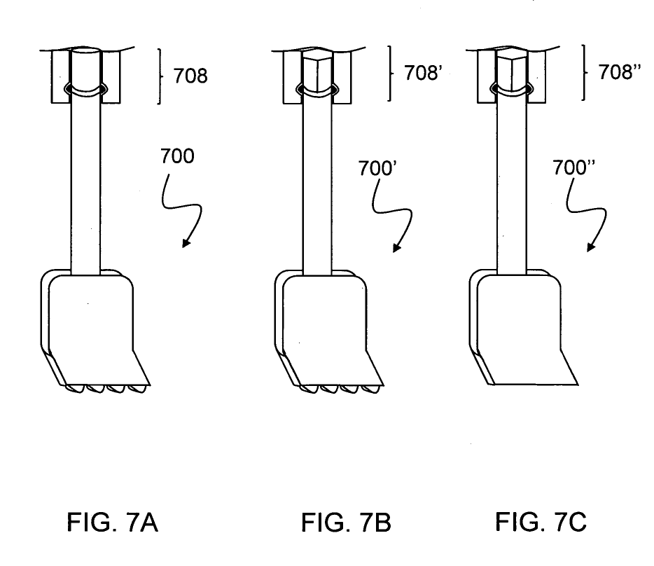

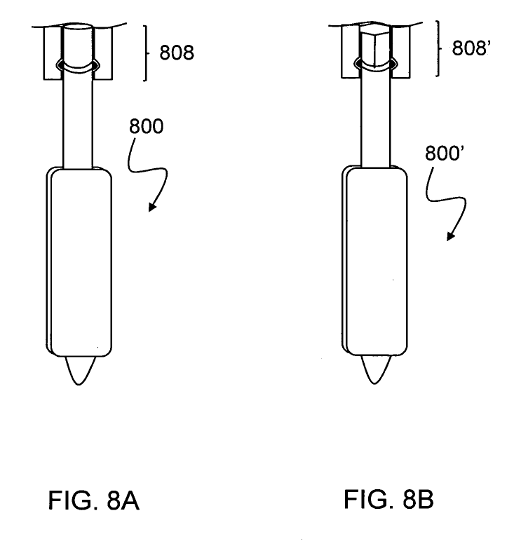

FIGS. 7A, 7B, 7C, 8A and 8B illustrate a portion of an instrument handle with other example retractor tools that may be used in connection with the system described herein such as a Richardson retractor 700, 700′, as shown in FIGS. 7A and 7B (e.g., narrow Richardson retractor that is approximately 1.5 inches wide or a broad Richardson retractor tool that is approximately 2.5 inches wide) and a spiked retractor tool 800, 800′ shown in FIGS. 8A and 8B.

As shown in FIGS. 7A and 8A, the retractors 700, 800 are illustrated, respectively, attached to portions of multiple-tool receptors 708, 808. In an embodiment, as shown, the attachment of the tool to the multiple-tool receptor may be via a Trinkle shank mechanism. The portions of the tools shanks of the retractors 700, 800 inserted into the multiple-tool receptors 708, 808 are illustrated as cylindrical. The retractors 700, 800 may be removably interchanged with an instrument handle according to the system described herein. Other designs for tool shanks and attachments mechanisms may be used in connection with the described system as further discussed elsewhere herein.

As shown in FIGS. 7B, 7C, and 8B, the retractors 700′, 700″, 800′ are illustrated respectively, attached to portions of multiple-tool receptors 708′, 708″, 808′. In an embodiment, as shown, the attachment of the tool to the multiple-tool receptor may be via a Trinkle shank mechanism. The portions of the tool shanks of the retractors 700′, 700″, 800′ inserted into the multiple-tool receptors 708′, 708″, 808′ are illustrated as hexagonal. Having a non-cylindrical tool shank insertion, such as hexagonal and/or other non-cylindrical cross-section, including octagonal and/or polygonal shape, may help reduce and/or prevent undesired rotation of the tool shank. Further, the tool shank may be inserted in multiple orientations which may be beneficial according to the type of surgical tool being used. For example, for a hexagonal shape, six different orientations of the tool may be possible. The portion of the cavity in the multiple-tool receptors 708′, 708″, 808′, which may lead into the cavity of the grip, as further discussed elsewhere herein, may also have the shape of the tool shank, such as a hexagonal shape. In other embodiments, and again depending on the type of surgical tool being used, rotation of the tool shank may be desired, which rotation may be facilitated by a cylindrical tool shank as discussed in connection with FIGS. 7A and 8A. The retractors 700′, 700″, 800′ may be removably interchanged with an instrument handle according to the system described herein. Other designs for tool shanks and attachments mechanisms may be used in connection with the described system as further discussed elsewhere herein. For example, it may be possible to provide a screw-like end in a tool shank in combination with a round ended tool shank and/or a non-round ended tool shank (e.g., hexagonal shank). The screw-like end may screw into the handle.

In an embodiment of the system described herein, a surgical kit prepared according to the system described herein may include an instrument handle and multiple tool components for being removably and interchangeably inserted into the instrument handle as further discussed elsewhere herein. In another embodiment, tools may be permanently attached to the instrument handle, as further discussed elsewhere herein, and a surgical kit may include multiple instruments having instrument handles with permanently attached tools. Any combination of the above in a surgical kit may also be possible.

Other tools may include clasps and clamps that may help in management and mitigation of fractures during surgical procedures. Further, other tools may be used with the system described herein for cutting and/or reaming procedures.

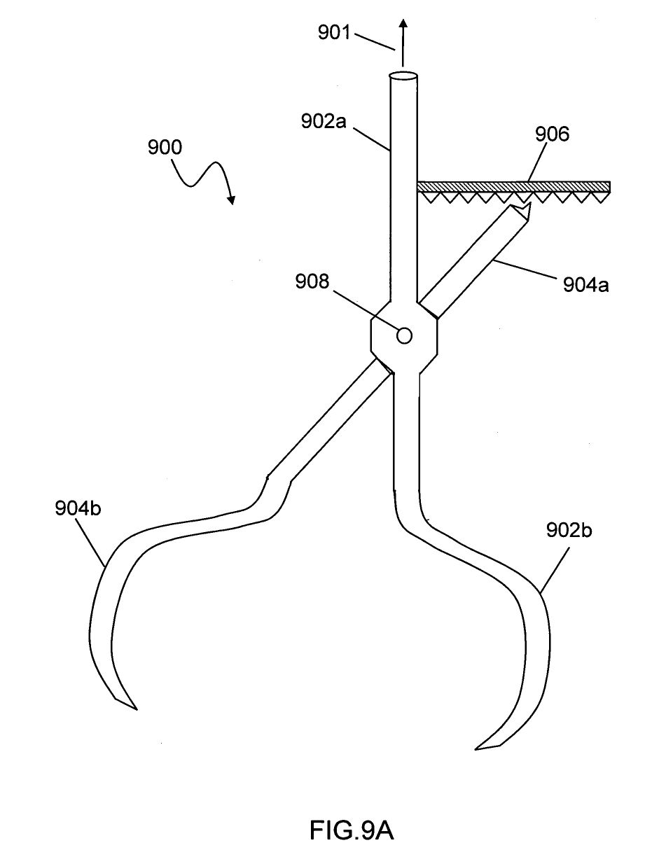

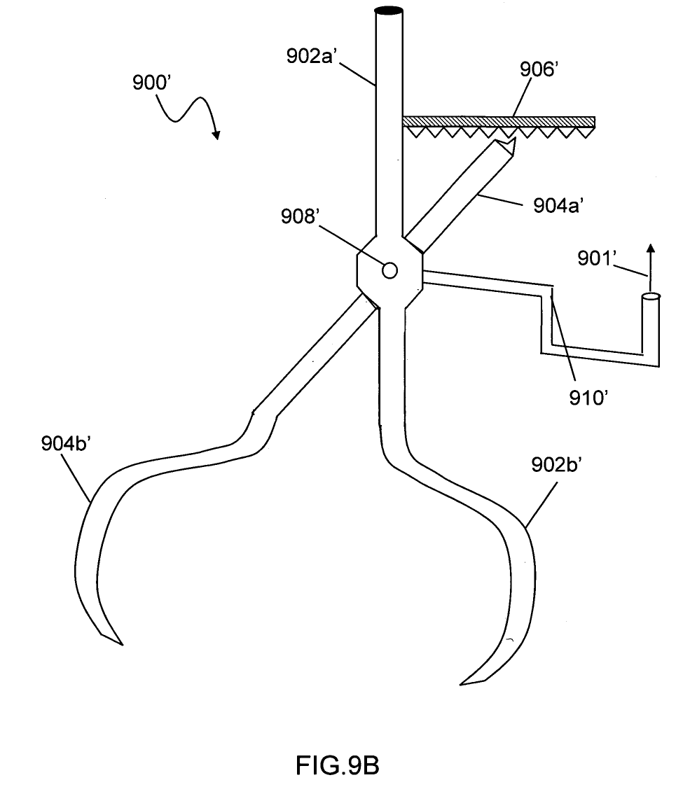

FIGS. 9A and 9B are schematic illustrations of a femoral grasp tool 900, 900′ that may be used with one or more of the instrument handles discussed in accordance with the system described herein. As shown in FIG. 9A, the femoral grasp tool 900 has an attachment leg 902a for attachment to the multiple-tool receptor, such as multiple receptor 108 of instrument handle 100 discussed elsewhere herein, in the direction of an arrow 901 as illustrated. A grasping arm 902b is coupled to the attachment leg 902a, and a grasping arm 904b is coupled to a leg 904a, which may pivot with respect to one another about a joint 908. The leg 904a may be manipulated into a locked position using a ratchet assembly 906 that may lock the leg 904a and grasping arm 904b into a desired position with respect to the attachment leg 902a and the grasping arm 902b. In accordance with the system described herein, a doctor using the instrument handle 100, into which the leg 902a of the femoral grasp tool 900 is inserted and attached, may be to able to exert greater force with enhanced control on the object being grasped by the femoral grasping tool 900. Other components and tools may also be included, for example, a muscle retractor and/or a slotting cutting guide and/or nerve protector may be coupled to one or more of the legs 902a, 904a and/or grasping arms 902b, 904b for moving muscle away, cutting a femur precisely and/or protecting the sciatic nerve from a cut.

As shown in FIG. 9B, the femoral grasp tool 900′ has a leg 902a′ coupled to a grasping arm 902b′ and a grasping arm 904b′ is coupled to a leg 904a′, which may pivot with respect to one another about a joint 908′. The leg 904a′ may be manipulated into a locked position using a ratchet assembly 906′ that may lock the leg 904a′ and grasping arm 904b′ into a desired position with respect to the leg 902a′ and the grasping arm 902b′. The legs 902a′ and 904a′ may be squeezed together by a user. An attachment leg 910′ may extend from, and in some embodiments through, the pivot 908′ and be suitable for insertion into an instrument handle in the direction of an arrow 901′, as illustrated, in accordance with the system described herein, such as instrument handle 100, for example. In accordance with the system described herein, a doctor using the instrument handle 100, into which the attachment leg 910′ of the femoral grasp tool 900′ is inserted and attached, may be to able to exert a greater lifting force on a femor and/or other object, with enhanced control on the object being grasped.

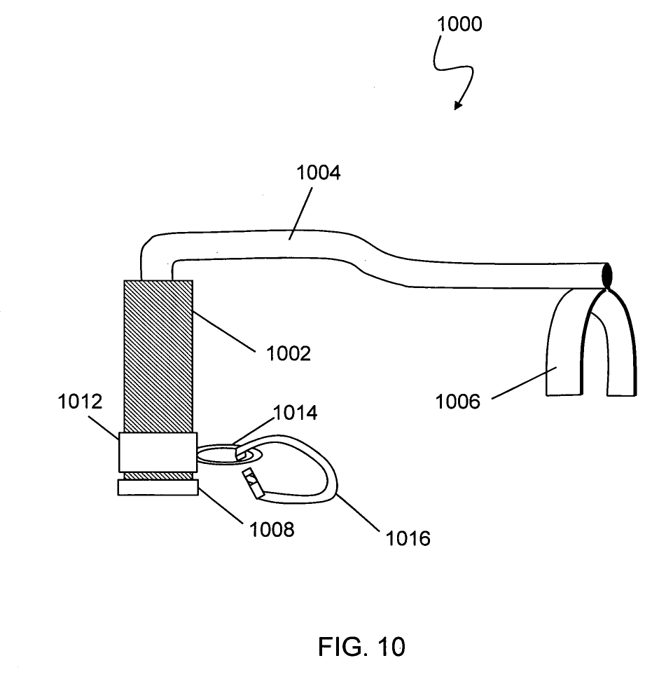

FIG. 10 is schematic illustration of a swivel mechanism 1110 for use with an instrument handle 1000 in accordance with the system described herein. The instrument handle 1000 may include a grip 1002, extension arm 1004, support brace 1006 and multiple-tool receptor 1008 that function like similar components 102, 104, 106 and 108 discussed elsewhere herein. The swivel mechanism 1110 may include a bushing 1112 positioned on the grip 1002 and coupled to an attachment opening 1114, such as a washer and/or other device that provides hole for attachment. A body linkage device, such as a strap, rope, harness and/or other flexible or inflexible support, and/or an attachment mechanism therefore, that is suitable for being worn on or around the body, may be attached to the opening 1114, as further discussed elsewhere herein. For example, a closed hook attachment component 1116, may be used that may be part of the body linkage device and attaches to the strap and/or other support component which may be wrapped around a user's body, e.g., waist, chest and/or neck, as discussed below. In this way, the amount of force and degree of control that a user may exert with the instrument handle 1000, and accordingly to the tool fitted therein, may be significantly enhanced. Other types of body supports and attachment mechanisms may be used in connection with the system described herein.

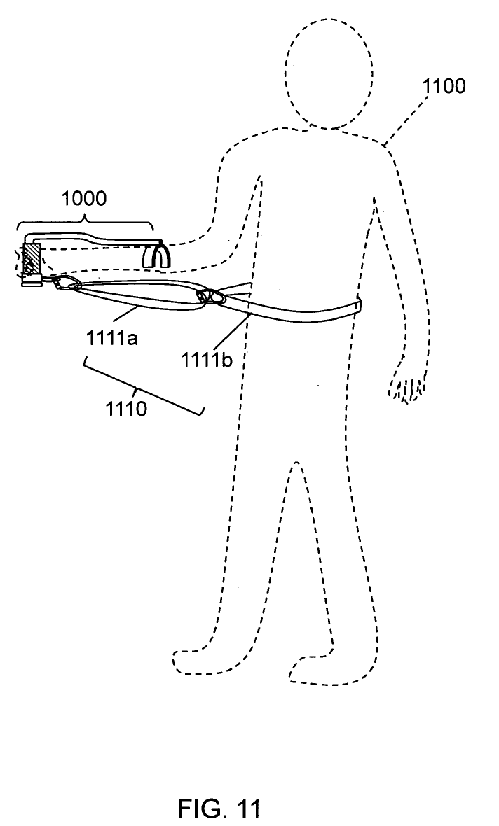

FIG. 11 is a schematic illustration of showing a user 1100 operating the instrument handle 1000 and including a body linkage device 1110 wrapped around the body of the user 1100. As further discussed elsewhere herein, the body linkage device 1110 may include suitable components for removably attaching the handle 1000 to the body of the user 1100, such as a first linkage device 1111a that is coupled to the handle 1000 and a second linkage device 1111b that is wrapped around the body, such as a strap. Other body linkage and support devices may be used in connection with the described system, as further discussed elsewhere herein. In an embodiment, the body linkage device may be a disposable component, such as disposable strap that may discarded after use in a procedure.

Various embodiments described herein may be combined together in accordance with the system described herein. For example, different styles of support brace, grip shape, grip angle, extension mechanisms and/or other components may be combined. The system described herein may be used for any other appropriate surgical tools, including knee and shoulder surgical tools used, for example, for knee and shoulder replacement surgery, a femoral tool with a screw head, etc. Further, in addition to surgical instruments, the instrument handle described herein may also be used in connection with other tools that involve application of a directional force, such as application of a downward pressure or pulling pressure. For example, the instrument handle may be used in connection with garden tools, such as tools for hand-tilling and hand-toweling, and with construction tools, such as a tool for ripping up shingles from a roof.

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

What is claimed is:

1. An instrument handle, comprising:
a grip;
an extension arm coupled to a first end of the grip;
a support brace coupled to the extension arm;
a multiple-tool receptor coupled to a second end of the grip, wherein the multiple-tool receptor includes an interface for removably attaching a tool, wherein the interface is coupled to the second end of the grip at a position opposite from the first end of the grip coupled to the extension arm.

2. The instrument handle according to claim 1, wherein the interface has a tool-receiving axis that is substantially perpendicular to the extension arm.

3. The instrument handle according to claim 1, wherein the support brace has a U shape.

4. The instrument handle according to claim 1, wherein the extension arm is adjustable in length.

5. The instrument handle according to claim 1, wherein at least one arm of the support brace is adjustable in length.

6. The instrument handle according to claim 1, wherein the support brace is movable on the extension arm.

7. The instrument handle according to claim 1, wherein the multiple-tool receptor includes at least one of: a bushing; a quick connect mechanism; a shank; and a chuck.

8. The instrument handle according to claim 1, further comprising:
a swivel mechanism coupled to the grip, the swivel mechanism including a opening for coupling to a body linkage device.

9. The instrument handle according to claim 1, wherein the grip is angled with respect to the extension arm.

10. A surgical instrument, comprising:
an instrument handle; and
a tool coupled to the instrument handle, wherein the instrument handle includes:
a grip;
an extension arm coupled to a first end the grip;
a support brace coupled to the extension arm, wherein the tool is coupled to a second end of the grip at a position opposite from the first end of the grip coupled to the extension arm.

11. The surgical instrument according to claim 10, further comprising:
a multiple-tool receptor coupled to the second end of the grip, wherein the multiple-tool receptor includes an interface for removably attaching the tool.

12. The surgical instrument according to claim 10, wherein the tool includes a femoral distractor hook.

13. The surgical instrument according to claim 10, wherein the tool includes a femoral grasp.

14. The surgical instrument according to claim 10, wherein the tool includes a retractor.

15. The surgical instrument according to claim 10, wherein the instrument handle further comprises:
a swivel mechanism coupled to the grip, the swivel mechanism including a opening for coupling to a body linkage device.

16. The surgical instrument according to claim 10, wherein the interface has a tool-receiving axis that is substantially perpendicular to the extension arm.

17. The surgical instrument according to claim 10, wherein at least one of the extension arm and the support brace are adjustable in length or position.

18. A method for performing a hip replacement procedure, comprising:
positioning a user's forearm in an instrument handle of a surgical instrument, the instrument handle including a grip, an extension arm a support brace that contacts the forearm, wherein a first tool of the surgical instrument extends from the grip in a direction substantially perpendicular to the extension arm;
exerting force on the surgical instrument in a direction substantially parallel to the extension arm, wherein the support brace braces against the forearm as a result of the force exerted.

19. The method according to claim 18, further comprising:
removing the first tool from the grip of the instrument handle and inserting a second tool into the grip of the surgical handle.

20. The method according to claim 18, wherein the bracing of the support brace against the forearm minimizes angulation and rotation of the surgical instrument.
